(19) United States
(12) Patent Application Publication
(10) Pub. No.: US 2023/0341343 A1
(43) Pub. Date: Oct. 26, 2023

(54) GAS DETECTION DEVICE AND GAS DETECTION PROCESS WITH A SENSOR AND WITH AN OXIDIZER

(71) Applicant: Dräger Safety AG & Co. KGaA, Lübeck (DE)

(72) Inventors: Malte BAESLER, Lübeck (DE); Tom PÖTHIG, Lübeck (DE)

(21) Appl. No.: 18/302,055

(22) Filed: Apr. 18, 2023

(30) Foreign Application Priority Data
Apr. 20, 2022 (DE) 10 2022 109 534.7
Jul. 1, 2022 (DE) 10 2022 116 509.4

(51) Int. Cl. G01N 27/04; G01N 1/44; G01N 33/00; G01N 31/12

(52) U.S. Cl. CPC G01N 27/04; G01N 1/44; G01N 33/0027; G01N 31/12

(57) ABSTRACT

A gas detection device (100) and a process monitor an area for a combustible target gas. A gas sample (G) enters into a measuring chamber (9). A semiconductor sensor (1) including an electrically conductive sensor component (10) has an electrical detection variable that changes with decreasing concentration of the combustible target gas. An oxidation component (2) oxidizes combustible target gas in the measuring chamber. The detection variable is measured at a detection time, and the oxidation component is in a switched-on state or is switched on. The oxidation component oxidizes at least a part of the combustible target gas in the measuring chamber up to a reference time. The detection variable is measured again at the reference time. The difference between the measured value at the reference time and the measured value at the detection time is an indicator for the concentration of combustible target gas.

### CROSS REFERENCE TO RELATED APPLICATIONS

This application claims the benefit of priority under 35 U.S.C. § 119 of German Application 10 2022 109 534.7, filed Apr. 20, 2022, and of German Application 10 2022 116 509.4, filed Jul. 1, 2022, the entire contents of which are incorporated herein by reference.

### TECHNICAL FIELD AND BACKGROUND

The present invention pertains to a gas detection device and to a process for monitoring a spatial area for a combustible target gas.

### SUMMARY

The present invention is used in one application to detect the presence of a combustible target gas, for example, methane (CH4), even when the concentration of the target gas is low, for example, below 10 ppm.

A basic object of the present invention is to provide a gas detection device and a gas detection process, which are capable of detecting a combustible target gas with a high level of reliability even when the target gas is present at a relatively low concentration and/or varying ambient conditions may have an effect on the measurement in a relevant manner.

The object is accomplished by a gas detection device having device features according to the invention and by a gas detection process having process features according to the invention. Advantageous embodiments of the gas detection device according to the present invention are, where appropriate, also advantageous embodiments of the gas detection process according to the present invention and vice versa.

The gas detection device according to the present invention and the gas detection process according to the present invention are capable of monitoring a spatial area for at least one combustible target gas, for example, for methane (CH₄). The process according to the present invention is carried out with the use of a gas detection device according to the present invention. The spatial area is, for example, an area of a production plant or a mine or the interior of a building or of a vehicle or of an aircraft.

The “combustible target gas” will be used below. It is possible that the gas sample in the measuring chamber comprises a plurality of combustible target gases simultaneously. The term “combustible target gas” shall also cover the situation in which different combustible target gases are present in the measuring chamber. The term “detection of a target gas,” which will be used below, comprises the process of detecting the presence of at least one target gas. The particular combustible target gas or particular combustible target gases that shall be detected is specified in one embodiment. Each combustible target gas shall be detected in the spatial area in another application.

The gas detection device comprises a measuring chamber. The gas detection device is configured in such a way and the process comprises the step that a gas sample flows continuously or at least from time to time from the area to be monitored into the measuring chamber, for example, by suctioning and/or by diffusion.

An electrically conductive sensor (an electrically conductive sensor unit) as well as an oxidizer (an oxidation unit) are arranged in or at the measuring chamber. The electrically conductive sensor has a measurable electrical detection variable and comes into an areal contact with the gas sample in the measuring chamber. This contact has an effect on the measurable electrical detection variable, especially on the electrical resistance of the electrically conductive sensor, as follows: In a first embodiment, the value of the detection variable of the electrically conductive sensor increases with decreasing concentration of the combustible target gas in the gas sample, which gas sample is located in the measuring chamber. In a second embodiment, the value of the detection variable decreases with decreasing concentration of the combustible target gas in the gas sample, which is located in the measuring chamber. In each case, the value of the detection variable of the electrically conductive sensor changes with decreasing concentration of the combustible target gas in the gas sample. In both embodiments, the detection variable is correlated with the target gas concentration in the gas sample. It is, of course, possible that the gas sample contains no target gas at all and does therefore not have an effect on the detection variable in a relevant manner or does not change the detection variable compared with a reference state.

The oxidizer can be switched on for a use of the gas detection device and optionally switched on and switched off during the use. The oxidizer in the switched-on state is capable of oxidizing combustible gas in the measuring chamber, but it can, of course, do so only when combustible gas is present in the measuring chamber.

Note: The terms “switching on” and “switching off” used above and hereinafter may designate an abrupt or even a gradual transition from a switched-off state to a fully switched-on state.

The gas detection device further comprises a detection sensor. The detection sensor is capable of measuring an indicator for the detection variable of the electrically conductive sensor, for example, an indicator for the current electrical resistance or for the electrical voltage that is present at the electrically conductive sensor, or for the intensity of the current that is flowing through the electrically conductive sensor, or for the entire electrical charge.

The gas detection device comprises, in addition, a signal-processing analysis unit, which is capable of receiving and processing a signal from the detection sensor. In one embodiment, the gas detection device comprises a housing, and the rest of the units just mentioned are located in the interior of the housing. In another embodiment, the analysis unit is arranged outside of the housing, and the signal of the detection sensor is transmitted, preferably in a wireless manner, to this analysis unit located at a distance.

The gas detection device is configured to carry out the following steps, and the process according to the present invention comprises the following steps:
The oxidizer is switched on or is in a switched-on state. The oxidizer in the switched-on state oxidizes the combustible target gas or each combustible target gas in the measuring chamber entirely or at least partially during an oxidation time period. The process of oxidation of the combustible target gas by the oxidizer reduces the quantity of combustible target gas in the measuring chamber and it optionally but not necessarily eliminates all combustible target gas in the measuring chamber. It is, of course, possible that no combustible target gas was present in the measuring chamber already at the beginning of the oxidation time period, especially when the area to be monitored currently does not contain any combustible target gas.
The detection sensor measures the detection variable of the electrically conductive sensor, more precisely an indicator for the detection variable at the respective times, namely at a detection time and at a reference time. The detection time is located chronologically before or even after the reference time. Due to the fact that the indicator is measured at two different times, two measured values of the detection variable are available (more precisely, there are two measured values for the indicator for the detection variable), and the two measured values pertain to two different times.
The gas detection device is operated such that the following situations will occur: When combustible target gas is present in the area to be monitored, combustible target gas is also present in the measuring chamber at the detection time. The target gas concentration in the measuring chamber may be just as high as or lower than in the area to be monitored. Thanks to the oxidation by the oxidizer, less combustible target gas is present or even no combustible target gas is present at all in the measuring chamber at the reference time even if combustible target gas is present in the area. If combustible target gas is present in the area to be monitored, a lower concentration of combustible target gas is present at the reference time in the measuring chamber than in the area and at the detection time in the measuring chamber, or even no combustible target gas is present at all, provided that combustible target gas is present in the area. This effect is achieved thanks to the oxidation during the oxidation time period.
The oxidizer is in a switched-on state at least during the oxidation time period and it is capable of oxidizing combustible target gas in the measuring chamber. In a first alternative, the detection time is at the beginning or before the beginning of the oxidation time period, and the reference time is at the end or after the end of the oxidation time period. In a second alternative, the reference time is, conversely, at the beginning or before the beginning of the oxidation time period, and the detection time is at the end or after the end of the oxidation time period. If the oxidizer is continuously operated in the switched-on state, the time period from the earlier to the later of the two times is used as the oxidation time period.
The analysis unit calculates the difference between the measured value for the detection variable of the sensor, which value was measured by the detection sensor at the detection time, and the measured value for the detection variable, which value was measured by the detection sensor at the reference time. Combustible target gas is present in the measuring chamber at the detection time if combustible target gas appears in the area to be monitored. Less combustible target gas or even no combustible target gas at all is present at the reference time.
In a first alternative, the analysis unit decides automatically, depending on this difference of the measured values, whether a combustible target gas with a concentration above a threshold is present in the gas sample or not. This decision depends, in addition, on whether the detection variable increases or decreases with rising concentration of the target gas, and/or from the amount of the difference, i.e. |dist|. In a second alternative, the analysis unit automatically determines, at least approximately, the concentration of the combustible target gas in the gas sample depending on the difference of the measured values. In the second alternative, the analysis unit preferably applies a stored relationship between the difference of the measured values and the target gas concentration to the calculated difference. At least in the second alternative, the oxidizer is preferably in the switched-off state at the detection time.
The two alternatives, i.e., the detection of the combustible target gas and the determination of the concentration of the combustible target gas, may be combined with one another.

According to the first embodiment, the detection variable of the electrically conductive sensor increases with decreasing concentration of the target gas in the measuring chamber. The measured value of the detection variable is therefore higher at the reference time than the measured value of the detection variable at the detection time, providing that a sufficient concentration of combustible target gas is present in the area to be monitored and therefore in the measuring chamber as well. According to the second embodiment, the measured value of the detection variable is correspondingly lower at the reference time than at the detection time.

If no combustible target gas is present in the area to be monitored and therefore in the measuring chamber as well, the two measured values of the detection variable, which were measured at the two times, are ideally equal. In practice the difference may differ zero due to different ambient conditions, even if no combustible target gas is present in the area to be monitored and hence the measuring chamber also fails to contain any combustible target gas at the detection time. However, this difference (the amount |dist|) between the two measured values is lower, as a rule, in the absence of target gas than the difference that results from the fact that combustible target gas is present in the measuring chamber at the detection time and the oxidizer oxidizes at least a part of this combustible target gas in the measuring chamber during the oxidation time period. Even when the oxidizer oxidizes only a part, but not all the combustible target gas in the measuring chamber, the difference is, as a rule, greater than when no combustible target gas is present in the area and therefore also in the measuring chamber. A difference that is not equal to zero or is outside of a predefined tolerance range is therefore a relatively reliable indicator that a combustible target gas is present in the area to be monitored and hence at the detection time as well in the measuring chamber .

The detection variable of the electrically conductive sensor frequently changes in a measurable manner due to the contact with combustible target gas, even if combustible target gas is present in the measuring chamber at a low concentration only. However, the detection variable depends, as a rule, not only on the concentration of combustible target gas, but also on ambient conditions, especially on the ambient temperature and the ambient humidity, optionally the ambient pressure. The value that the detection variable assumes in a state in which no combustible target gas is present in the measuring chamber is often also called zero value (reference value). Gas detection devices known from the state of the art with a sensor, which sensor has a detection variable that responds to the concentration of combustible target gas, frequently have the following drawbacks: The zero point depends strongly on ambient conditions and is, as a rule, unknown. Or else, a sensor is needed for a relevant ambient condition, i.e. an additional sensor.

The present invention solves this problem by the oxidizer oxidizing the combustible target gas in the measuring chamber during the oxidation time period, and the current zero point is therefore measured for the detection variable of the electrically conductive sensor at the reference time. The term “current zero point” suggests that the zero point depends, as a rule, on varying ambient conditions and can therefore vary. The time period between the two times is usually so short that the ambient conditions do not change substantially during this time period and the measured value obtained at the reference time can therefore be used as the zero point for the measurement at the detection time despite the time interval. The difference calculated according to the present invention shows a sufficiently accurate agreement with the difference between the measured value obtained at the detection time and the (actual) zero point occurring at the detection time.

The present invention avoids the need to have to adjust the gas detection device anew before each use in order to find an actual zero point. As is being described, the zero point is rather obtained automatically, at least approximately, by the measurement performed at the reference time. The present invention also eliminates in many cases the need for the gas detection device to comprise a sensor for an ambient. The present invention also avoids the need to calibrate the gas detection device during a use in order to adapt the gas detection device to a possible amendment or drift of the zero point. In particular in a situation where the ambient condition changes very quickly and/or the concentration of the target gas quickly varies, the result of a calibration, i.e. an adaption of the zero point, can be outdated and not be valid due to this rapid change. The gas detection device according to the invention, however, yields in many cases reliable results even in the presence of quickly varying ambient conditions or target gas concentration. Some reasons: The difference between the two time points can be selected sufficiently short. The measuring and evaluation result only depends on the result between the two measured indicator values but not from a zero point which was fixed in advance. According to the invention the determination about the presence of target guards is made on the basis of a difference between two actual measured values of the detection variable. In many cases this feature enables to reliably detect target gas with a small concentration, and false alarms are in many cases avoided. This effect would often not be reliably achieved if the precise knowledge of a zero point were required.

According to the present invention, the electrically conductive sensor has an electrical detection variable, which increases in a first embodiment and decreases in a second embodiment (in both embodiments changes) with decreasing target gas concentration. This detection variable is in particular the electrical resistance in one embodiment, an electrical capacity (capacitance) in another embodiment and the electrical potential in a third embodiment. The electrically conductive sensor comprises, for example,
an electrical semiconductor, whose electrical resistance depends on the target gas concentration, or
a heat tone sensor, whose temperature depends on the target gas concentration, or
a photoelectric sensor, which generates an electrical signal as a function of the intensity of occurring electromagnetic radiation, wherein this electromagnetic radiation is attenuated by target gas,
a photoionization detector, which generates an electrical signal as a function of an ionization,
a photoacoustic sensor, which generates an electrical signal as a function of an acoustic effect, which depends on the target gas concentration, or
an electrochemical sensor, which generates an electrical current, whose intensity and/or voltage depends on the target gas concentration.

According to the present invention, the oxidizer oxidizes in the course of the oxidation time period at least a part of the combustible target gas in the measuring chamber. This oxidized part is preferably at least 30%, especially preferably at least 50%, and especially at least 80% of the quantity of combustible target gas that is present in the measuring chamber at the beginning of the oxidation time period. In one embodiment, the oxidizer oxidizes all the combustible target gas in the measuring chamber during the oxidation time period.

In one embodiment, the gas detection device additionally comprises a heating element. This heating element is capable of heating a gas sample in the measuring chamber. The heating element can be switched on and switched off again and can therefore selectively be operated in a switched-on state or in a switched-off state. According to this embodiment, the oxidizer can also be switched on and switched off and can therefore selectively be operated in a switched-on state or in a switched-off state. According to the embodiment with the heating element, the process additionally comprises the following steps, and the gas detection device is configured to carry out the following additional steps:
The heating element is switched on at the beginning of a heating time period. The heating element is switched off again at the end of the heating time period.
The temperature of the heating element in the switched-on state rises.
The heating element heats the gas in the measuring chamber during the heating time period.
During the heating time period the oxidizer is in the switched-off state.

Conversely, the heating element is in the switched-off state at least during the oxidation time period. Either the oxidizer or the heating element is in the respective switched-on state at each time, but the two units are never at the same time in the switched-on state.

According to the present invention, the target gas concentration being sought has an effect on the detection variable of the electrically conductive sensor. The detection variable additionally depends, as a rule, on the temperature of the electrically conductive sensor. The oxidizer in the switched-on state as well as the step of switching on and switching off the oxidizer have an effect on this temperature. The embodiment with the heating element reduces the effect of the oxidizer onto the electrically conductive sensor as the oxidizer in the switched-on state inputs more thermal energy then in the switched-off state. The reliability of the detection result is increased by the reduction of the effect. Ideally The switched-on heating element and the switched-on oxidizer bring about the same input of thermal energy per unit of time into or onto the electrically conductive sensor.

According to the embodiment just described, the heating element is in the switched-on state during the heating time period and is in the switched-off state during the oxidation time period, preferably during the entire oxidation time period. During the oxidation time period the oxidizer is in the switched-on state, and it is in the switched-off state at least during a segment of the heating time period, preferably during the entire heating time period. Consequently, the oxidation time period and the heating time period preferably do not overlap at all or do so at a time only. It is also possible that neither the oxidizer nor the heating element are in the switched-on state during a further time period.

In one embodiment, the end of the heating time period coincides with the beginning of the oxidation time period. Or else, the end of the oxidation time period coincides with the beginning of the heating time period. These two embodiments reduce the effect of the temperature of the oxidizer on the electrically conductive sensor compared to an embodiment in which the heating element is switched off, after which a time period elapses, and the oxidizer is switched on only thereafter, or vice versa. Compared to an embodiment in which the heating element and the oxidizer both are in the switched-on state from time to time, this embodiment saves electrical energy. In addition, it is easier to maintain the input of thermal energy per unit of time into the electrically conductive sensor at a constant value.

Preferably the gas detection device is always in exactly one of the following states during a use:
The oxidizer is in the switched-on state, and the heating element is in the switched-off state.
The heating element is in the switched-on state, and the oxidation component is in the switched-off state.
The measuring chamber is flushed, or a gas sample flows into the measuring chamber. Preferably both the heating element and the oxidizer are then in the switched-off state.

When not in use, the gas detection device may be switched off, i.e., it may be in a resting state.

As was already described, both the oxidizer switched-on state and the heating element switched-on in the state cause thermal energy to act on the sensor component. The input of thermal energy per unit of time, which is brought about by the switched-on heating element, is preferably exactly equal to the input of thermal energy per unit of time by the switched-on oxidizer.

The input of thermal energy per unit of time, which is brought about by the oxidizer , depends on the geometry, especially on the surface and on the temperature of the oxidizer as well as on the distance between the oxidizer and the electrically conductive sensor. If the input of thermal energy per unit of time is equal and no combustible target gas is present in the measuring chamber, the value of the detection variable is ideally equal as well; more precisely, if the oxidizer is in the switched-on state and the heating element is in the switched-off state, the detection variable ideally assumes the same value as with the oxidizer being in the switched-off state and with the heating element being in the switched-on state. This is optionally true only after a transient phase, which occurs when oxidizer or the heating element is switched on or switched off. In many cases this implementation of the heating element saves the need for controlling in a closed loop manner the temperature of the detection sensor or the oxidizer or the heating element.

According to the present invention, the oxidizer is in the switched-on state at least during the oxidation time period. In one embodiment, the oxidizer is in the switched-off state at least during an inlet time period. The oxidizer is switched on and/or switched off during the ongoing operation.

A fluid communication is established between the measuring chamber and the surrounding area at least during the inlet time period and the gas samples flows from the spatial area to be monitored into the measuring chamber, for example, by diffusion or suctioning. This inlet time period precedes the oxidation time period. Ideally the inlet time period does not overlap the oxidation time period or it does so only at one time. The detection time is outside of the oxidation time period and is preferably within the inlet time period, in the inlet time period especially preferably at the beginning of the inlet time period. Since the oxidizer is continuously in the switched-off state during the inlet time period or at least during most of the inlet time period, the oxidizer oxidizes no combustible target gas, or it oxidizes only a negligibly small quantity of target gas only. Combustible target gas will therefore collect in the measuring chamber during the inlet time period, providing that the area to be monitored contains combustible target gas.

The embodiment with the oxidizer, which is switched on or switched off during the ongoing operation, makes it possible in many cases for the measuring chamber to be continuously in a fluid communication with the area to be monitored. It is not necessary to open or to close a closure for an inlet to the measuring chamber during the ongoing operation. Especially in case of a relatively low concentration of combustible target gas, the switched-on oxidizer oxidizes combustible target gas in the measuring chamber very rapidly, so that the fluid communication does not result in any appreciable distortion of measurement results.

In one embodiment, the detection time is at the beginning of the oxidation time period. A time interval is present in a preferred embodiment between the inlet time period and the oxidation time period. There is a heating time period within this time interval. The optional heating element is preferably switched on at the end of the inlet time period and is switched off again at the end of the inlet time period, i.e., before or at the beginning of the oxidation time period.

According to the present invention, the oxidizer is in the switched-on state at least in the course of the oxidation time period and it oxidizes combustible target gas in the measuring chamber. The embodiment described below may be combined with an oxidizer that can be switched on and switched off. However, the embodiment described below eliminates the need to switch on and/or switch off the oxidizer during the ongoing operation. The oxidizer may also be continuously in the switched-on state during the ongoing use. According to this other embodiment, the measuring chamber may be operated selectively in an open state or in a closed state. When the measuring chamber is in the open state, the gas sample can flow from the area to be monitored into the measuring chamber. Consequently, a fluid communication between the measuring chamber and the area to be monitored is established in the open state. The measuring chamber is sealed (closed, separated) in a fluid-tight manner against the area in the closed state. “Fluid-tight” means aside from inevitable openings or gaps. The gas detection device is preferably switched off and is separated from the surrounding area in a fluid-tight manner in a resting state.

The gas detection device comprises in this embodiment a closable opening, for example, a valve or a recess with a flap or a diaphragm for the opening. When this opening is opened, a gas sample can flow from the surrounding area into the measuring chamber. When the opening is closed, the measuring chamber is sealed against the surrounding area, so that no gas sample can then flow into the measuring chamber. The opening is preferably closed during the oxidation time period and is opened at least from time to time before and/or after the oxidation time period. It is, however, also possible that gas can flow into the measuring chamber during the oxidation time period as well. The quantity of the target gas, which flows into the measuring chamber during the oxidation time period when the closure is opened, is, as a rule, smaller than the quantity that is oxidized by the oxidizer during the oxidation time period.

The measuring chamber is in the open state during an inlet time period. This inlet time period preferably includes the detection time, or the inlet time period precedes the detection time. The measuring chamber is preferably in the closed state at the reference time.

The measuring chamber is in the closed state at least during the oxidation time period, and the oxidizer oxidizes combustible target gas, ideally all the combustible target gas, in the measuring chamber. Since the measuring chamber is in the closed state, no combustible target gas can flow from the area into the measuring chamber. The measuring chamber is in the open state during the inlet time period already mentioned. Combustible target gas collects during this inlet time period in the measuring chamber, providing that combustible target gas is present in the area to be monitored.

These two embodiments may be combined, for example, as follows: During the oxidation time period the oxidizer is in the switched-on state, and the measuring chamber is in the closed state. During the inlet time period the oxidizer is in the switched-off state, and the measuring chamber is in the open state. In many cases this combination of the two embodiments makes it possible to detect the target gas with an even higher reliability, even when it is present only at a relatively low concentration in the area to be monitored. Optionally a heating time period occurs between the inlet time period and the oxidation time period.

In a first alternative of the present invention, the detection time precedes the reference time. A gas sample has flowed into the measuring chamber at the latest until the detection time. The oxidation time period begins at or after the detection time and ends before or at the reference time. The oxidizer is preferably switched on between the detection time and the reference time, and/or the measuring chamber is separated from the area to be monitored in a fluid-tight manner.

Conversely, the reference time precedes the detection time in the second alternative of the present invention. The oxidation time period begins at or after the reference time and ends before or at the detection time. In one embodiment, the measuring chamber is separated from the area in a fluid-tight manner at least during the oxidation time period. The oxidizer has oxidized combustible target gas in the measuring chamber until the reference time. The gas sample flows into the measuring chamber after the reference time and at least until the detection time, and optionally even thereafter. The oxidizer is preferably switched off between the reference time and the detection time, and/or a fluid communication is established between the measuring chamber and the area.

It is possible that the gas detection device is operated such that a plurality of oxidation time periods occur one after another, and there is a gap between two consecutive oxidation time periods and the oxidizer oxidizes target gas in the measuring chamber at least during each oxidation time period. The indicator for the detection variable is measured at a respective detection time each and at a reference time for each oxidation time period, and the analysis according to the present invention is carried out anew for each oxidation time period. Combustible target gas can in many cases be detected hereby relatively rapidly.

According to the embodiments described below the oxidizer can preferably be switched on and switched off. An oxidation time period is predefined as a fixed value in a first embodiment. The duration of the oxidation time period or of each oxidation time period is equal to this oxidation time period. The oxidation time period and hence each oxidation time period are as long as needed, on the one hand, and as short as possible, on the other hand. “As long as needed” means that the oxidizer oxidizes the combustible target gas in the measuring chamber even at the highest expected concentration of combustible target gas in the measuring chamber, so that the measuring chamber is free from combustible target gas at the end of the oxidation time period.

In a second embodiment, the duration of the oxidation time period or of at least one oxidation timer period, i.e., the time period during which the oxidizer is in the switched-on state, depends on the concentration of combustible target gas in the measuring chamber. The second embodiment eliminates the need to predefine an oxidation time period as a fixed value. According to the second embodiment, the gas detection device is additionally configured to carry out the following steps, and the process additionally comprises the following steps:

A slope calculation sequence is carried out at least once. The slope calculation sequence or each slope calculation sequence comprises the following steps:
The indicator for the detection variable is measured at at least two times, and these times are chronologically spaced apart from one another. Both times are within the oxidation time period. The indicator for the detection variable is preferably measured at the detection time and at at least one additional time, which additional time is chronologically after the detection time. A sampling rate is preferably predefined for the measurement of the detection variable, and this sampling rate determines the time interval between two measurement times directly following one another.
The time course of the detection variable during the oxidation is approximately determined by the measurements. If combustible target gas is present in the measuring chamber, the detection variable increases with time according to the first embodiment of the electrically conductive sensor until all target gas has been oxidized. The slope of the time course of the detection variable is consequently positive until all target gas has been oxidized. According to the second embodiment, the detection variable decreases, and the slope is negative until all target gas has been oxidized. If no combustible target gas is present in the measuring chamber, the detection variable remains approximately constant during the oxidation time period in both embodiments.
Depending on the measured values of the detection variable, an indicator for the slope of the detection variable is calculated, preferably by the analysis unit, for the derivation of the time course of the detection variable over time. In the simplest case, this indicator for the slope is the difference between the two measured values of the detection variable, which were measured at the two chronologically most recent times. The calculated slope may be variable over time.

If the value of the slope of the time course of the detection variable is below a predefined threshold, the measurement of the detection variable is ended. The chronologically most recent time is used as the reference time. The measured value at the chronologically most recent time is used as the measured value at the reference time. The threshold may be equal to zero or greater than zero. If the slope is below the predefined threshold, practically all the target gas present in the measuring chamber has been oxidized.

In the second embodiment, the duration of the oxidation time period does, as a rule, increase with increasing concentration of combustible target gas in the measuring chamber. The second embodiment will then lead in many cases to an especially rapid detection result when no combustible target gas is present in the area to be monitored and consequently neither in the measuring chamber. Only ambient conditions have an effect on the slope in this case, and the slope remains, as a rule, below the predefined threshold. On the other hand, the second embodiment leads to a reliable measurement result even when a very high concentration of combustible target gas is present. All combustible target gas in the measuring chamber is also oxidized, as a rule, in this case, so that the measured value at the second time (at the chronologically most recent time) acts reliably as a zero value in this case as well.

A functional relationship, which describes the time course of the detection variable during the oxidation time period, is predefined in a third embodiment. This functional relationship comprises at least one model parameter. This functional relationship often has the shape of an exponential function, i.e.,

f(t)=A−C*exp(−α*t) or f(t)=A*[1−C*exp(−α*t)]

with the model parameters A, C and α.

The detection variable (more precisely, the indicator for the detection variable) is measured several times during the oxidation time period, as a result of which a random sample is obtained. Values for the model parameters are calculated automatically by means of this random sample. The measured value for the second time can frequently be predicted with sufficient reliability by an extrapolation. The third embodiment often rapidly leads to a reliable measurement result both at a low target gas concentration and at a higher target gas concentration.

No fixed time period needs to be predefined in the third embodiment, either. It is also possible, however, to predefine a fixed number N>1 of measured values for the random sample, i.e., for the number of random sample elements, and to end the oxidation time period when the N measured values are available for the random sample.

The second embodiment and the third embodiment may be combined with one another.

In one embodiment, the oxidizer (oxidation unit, oxidation component) comprises the electrically conductive sensor. If the oxidizer is in the switched-on state, electrical current flows through the sensor component. With the oxidizer in the switched-off state, no electrical current flows through the sensor component. For example, the oxidizer is configured as a so-called pellistor and comprises a heating element, which acts as the sensor component. The oxidizer comprises, furthermore, a ceramic jacketing around the heating element and a catalytic coating on the ceramic jacketing or a catalytic admixture in the ceramic jacketing. The heating element of the pellistor acts as the electrically conductive sensor. The detection sensor measures a detection variable of the heating element.

The oxidizer and the electrically conductive sensor component are separated from one another in another embodiment. An electrical voltage can be applied to the sensor component, and electrical current will flow through the sensor component as a consequence of this, doing so independently from whether the oxidizer is in the switched-on or in the switched-off state. According to this other embodiment, the oxidizer is used only to oxidize combustible target gas possibly present in the measuring chamber, but not to additionally detect this target gas. The detection sensor preferably does not measure a variable of the oxidizer. The oxidizer may be configured as a pellistor in this other embodiment as well.

In one application, the spatial area to be monitored directly adjoins the gas detection device, and the gas sample can flow through an inlet into the measuring chamber. In another application, a distance is formed between the spatial area to be monitored and the gas detection device. The gas sample can flow only through a fluid-guiding unit (fluid conveying unit) from the spatial area and an inlet to the measuring chamber, but it cannot bypass this fluid-guiding unit. Thanks to the distance in space, the gas detection device is extensively protected from environmental effects in the area to be monitored. The fluid-guiding unit may have especially the form of a hose or a tube. The gas detection device preferably suctions a gas sample from the area to be monitored through the fluid-guiding unit.

In one embodiment, the fluid conveying unit is connected to an adapter in a fluid-tight manner. The adapter can be attached to the gas detection device and be removed again from the gas detection device. With the adapter attached, the gas sample can flow only through the fluid-guiding unit to the measuring chamber, and it can flow directly from the spatial area to the measuring chamber with the adapter removed.

The gas detection device according to the present invention may be configured as a portable device, in which case a user carries along this portable device. This portable device preferably comprises a separate power supply unit. The gas detection device according to the present invention may also be configured as a stationary device and be connected at least from time to time to a stationary power supply network. The gas detection device according to the present invention may comprise an output unit, and an alarm or the determined concentration can be outputted to this output unit.

The present invention will be described below on the basis of exemplary embodiments. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a first embodiment of the gas detection device according to the present invention;

FIG. 2 is a schematic view showing a second embodiment of the gas detection device according to the present invention;

FIG. 3 is a graph showing a sequence with measurements of the gas detection device as an example;

FIG. 4 is a perspective view showing the oxidation component as an example; and

FIG. 5 is an exemplary flow chart for the use of the gas detection device.

### DESCRIPTION OF PREFERRED EMBODIMENTS

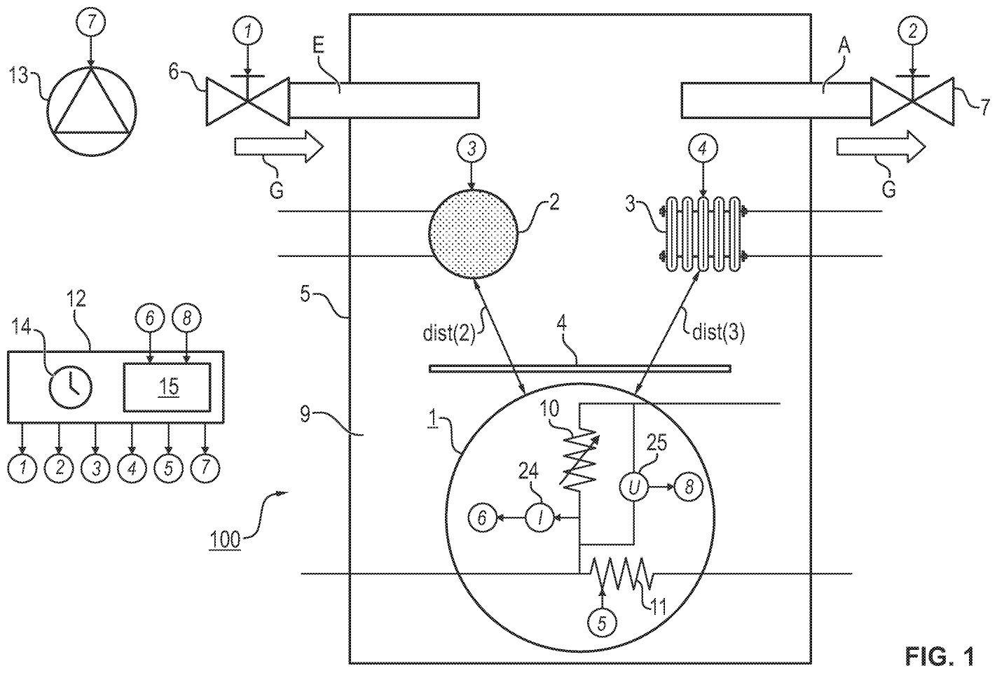

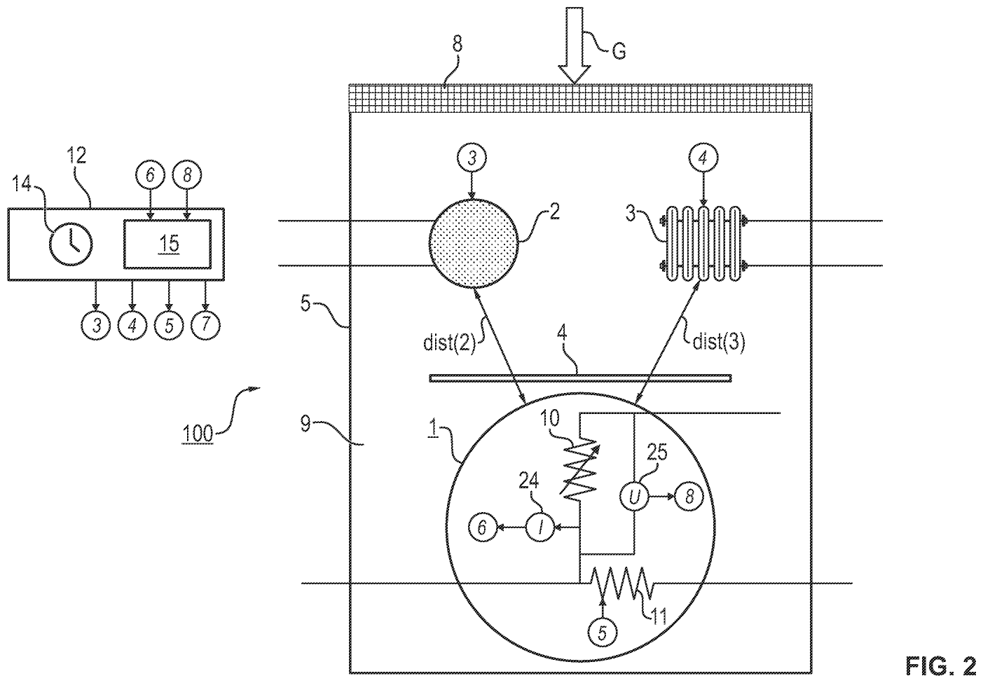

Referring to the drawings, FIG. 1 schematically shows a first embodiment of the gas detection device 100 according to the present invention, and FIG. 2 shows a second embodiment.

The gas detection device 100 is used in one application to monitor a spatial area for the presence of a combustible gas and to determine the concentration of a combustible gas. This combustible gas will hereinafter be called “target gas.” The spatial area is, for example, a refinery or another production plant, the interior of a building, a mine, a vehicle, or an aircraft. The combustible gas is, for example, methane (CH₄).

In another application, the gas detection device 100 is used to test a test subject for alcohol. The air that is exhaled by a test subject is known to contain breath alcohol if this test subject consumed alcohol and alcohol is therefore still present in his blood and/or in his mouth. The gas detection device 100 comprises a mouthpiece in this application. The test subject breathes into the mouthpiece, and at least a part of the breath sample given enters the interior of the gas detection device 100. Gaseous breath alcohol is consequently the combustible target gas in this application.

In one embodiment, the gas detection device 100 is a portable device, which a person can hold in his or her hand or which he can also attach to his/her clothing or protective equipment and which comprises a separate power supply unit. For example, a user carries along such a gas detection device 100 while he or she is in an area in which at least one combustible target gas may be present. Or else, a test subject takes the gas detection device 100 into one of his or her hands and gives a breath sample into the mouthpiece. The gas detection device 100 may also be configured as a stationary device, which can be connected to a stationary power supply network and does not necessarily have a separate power supply unit.

The gas detection device 100 comprises a housing 5, which encloses a measuring chamber 9, aside from the openings described below and aside from inevitable openings and slots, in a fluid-tight manner.

In the first embodiment, an inlet E in the housing 5 leads from the surrounding area into the measuring chamber 9, and an outlet A in the housing 5 leads from the measuring chamber 9 into the surrounding area. An actuatable valve 6 is capable of optionally releasing or blocking the inlet E. An actuatable valve 7 is capable of optionally releasing or blocking the outlet A. With the valve 6 open, a gas sample G can flow from the surrounding area, i.e., from the area to be monitored, through the inlet E into the measuring chamber 9. With the valve 7 open, the gas sample G can flow from the measuring chamber 9 into the surrounding area. A signal-processing control unit 12 with a system clock 14 is capable of automatically actuating the valves 6 and 7 as well as additional components described below.

The control unit 12 is optionally capable of actuating a pump 13, and the actuated and thereby activated pump 13 suctions the gas sample G through the open inlet E into the interior of the gas detection device 100. With the outlet A open, gas flows at the same time out of the measuring chamber 9. It is also possible that the gas sample G diffuses through the inlet E into the interior of the gas detection device 100.

Also possible is an embodiment in which the valves 6 and 7 are not present even though the pump 13, the inlet E and the outlet A are present. It is possible that the inlet E and the outlet A are continuously open. It is also possible that the valve 6 is replaced by another form of a closure, for example, by a perforated orifice with a hole pattern, wherein the hole pattern has at least one hole and wherein the perforated orifice is movable relative to the housing 5. Depending on the position of the perforated orifice relative to the housing 5, the hole or one hole of the hole pattern overlaps the inlet E, and the inlet E is open, or the perforated orifice closes the inlet E.

The pump 13 may be driven continuously or be switched on or switched off alternatingly during the operation of the gas detection device 100. The embodiment in which the pump 13 is switched on continuously may be combined with the movable perforated orifice. The embodiment in which the pump 13 is switched on and switched off avoids the need to provide a perforated orifice or another closure.

In the second embodiment (FIG. 2) a gas-permeable diaphragm 8 separates the measuring chamber 9 from the surrounding area, so that the gas sample G can flow from the surrounding area through the diaphragm 8 into the measuring chamber 9 and also again through the diaphragm 8 out of the measuring chamber 9. In the second embodiment shown, the diaphragm 8 replaces the inlet E and the outlet A of the first embodiment (FIG. 1). A pump 13 (not shown in FIG. 2) can suction the gas sample G in the second embodiment as well, this time through the diaphragm 8. A flame guard, not shown, for example, a metallic grid, preferably prevents flames from the measuring chamber 9 from entering into the surrounding area through the diaphragm 8.

A perforated orifice (not shown) may be movable relative to the housing 5 and separate or release the measuring chamber 9 in a fluid-tight manner against the surrounding area depending on the position of the measuring chamber 9 in the second embodiment as well.

Unless mentioned otherwise, the following description pertains to both embodiments.

A semiconductor sensor 1 is arranged in the interior of the measuring chamber 9. The semiconductor sensor 1 comprises a semiconductor component 10 as well as a heating element 11. The semiconductor component 10 acts as the electrically conductive sensor in the sense of the claims. The semiconductor component 10 is electrically conductive and is made preferably of a metal oxide, especially preferably of a semiconductor, for example, tin dioxide (SnO₂). The semiconductor component 10 is shown as a wire in FIG. 1 and FIG. 2, but should be considered to be a symbol in an equivalent circuit diagram and only one of several possible embodiments. Electrical contacts for the semiconductor component 10 and for other components of the gas detection device 100, which will be described below, are suggested.

A gas sample G in the measuring chamber 9 reaches the semiconductor component 10. The gas sample G acts on the semiconductor component 10 and has an effect on the electrical resistance R thereof. The electrical resistance R is consequently the affected and measurable detection variable in the exemplary embodiment. The chemical effect in the exemplary embodiment is such that the electrical resistance R of the semiconductor component 10 decreases with increasing concentration of the combustible target gas in the gas sample G and hence in the measuring chamber 9. One reason: A combustible target gas frequently has a higher coefficient of thermal conductivity than breathing air, and combustible target gas in the measuring chamber 9 therefore cools the semiconductor component 10.

A preferred manner of functioning of the semiconductor component 10 will be described below based on the example of tin oxide (SnO₂) as the material of the semiconductor component 10: The electrical conductivity and hence the electrical resistance R depend on the number of free electrons (charge carriers) in the semiconductor component 10. The surface of SnO₂ crystals has O₂ vacancies, so that the electrons, which make adjacent Sn atoms available, do not find a partner. These electrons are freely moving. The number of the freely moving electrons has an effect on the electrical conductivity and hence on the electrical resistance R of the semiconductor component 10. The semiconductor component 10 adsorbs on its surface oxygen from the surrounding area. As a result, at least a part of the O₂ vacancies is occupied due to adsorption of surrounding oxygen and the previously free electrons are bound. The semiconductor component 10 is operated in the heated state, for example, by means of the heating element 11. A combustible target gas is oxidized on the surface of the semiconductor component 10, and oxygen, which was adsorbed as was just described, is again desorbed. The density of charge carriers in the form of free electrons therefore increases again. The higher the percentage of oxygen in the area surrounding the semiconductor component 10, the larger is the amount of free electrons which are bound to oxygen and the higher is the electrical resistance R.

The oxidation of combustible target gas consequently decreases the quantity of oxygen, which the semiconductor component 10 can adsorb. Under otherwise constant ambient conditions, the electrical resistance R of the semiconductor component 10 therefore decreases with increasing concentration of a combustible target gas in the measuring chamber 9. This property is utilized according to the present invention.

An indicator for the current electrical resistance R of the semiconductor component 10 is measured again at each sampling time of a predefined sequence of sampling times. For example, a voltage sensor 25 measures the electrical voltage U, which is present on the semiconductor component 10. A current intensity sensor 24 measures the intensity I of the electrical current, which flows through the semiconductor component 10. The electrical resistance R=U/I is derived from the voltage U and the current intensity I. As was just described, this electrical resistance R is correlated with the concentration of the target gas in the measuring chamber 9 and depends, in addition, on the ambient conditions, especially on the temperature in the measuring chamber 9.

The heating element 11 has the form of an electrical resistor [in] the exemplary embodiment and is in thermal contact with the semiconductor component 10, so that the temperature of the heating element 11 is with sufficient accuracy equal to the temperature of the semiconductor component 10. The heating of the semiconductor component 10 causes, as was described above, the oxidation of combustible target gas and the desorption of oxygen.

Variable ambient conditions, especially temperature, humidity, and air pressure, likewise have an effect on the electrical conductivity of the semiconductor component 10. A possible cause is that ambient conditions can change the surface temperature of the semiconductor component 10, for example, the temperature of the outer surface of the semiconductor sensor 1.

How the effect of these ambient conditions on the electrical resistance R is compensated by calculation up to a certain degree will be described below. Since this effect is compensated, measured values of the electrical resistance R, generally of the detection variable, can be used to determine the target gas concentration being sought.

The temperature of the semiconductor component 10 is maintained at a constant value, specifically at a temperature above each ambient temperature possible during the use, in a preferred embodiment of the present invention. The effect of the ambient temperature on the electrical resistance R of the semiconductor component 10 is reduced thereby.

The control unit 12 preferably regulates the temperature of the heating element 11 with the regulation target that the temperature of the heating element 11 shall remain constant despite variable ambient conditions and that the input of thermal energy per unit of time, which the heating element 11 applies to the semiconductor component 10, shall therefore likewise remain constant, even at varying ambient temperature. To change the thermal energy that is released by the heating element 11 when a change is needed, the control unit 12 changes the electrical voltage U that is present on the heating element 11 in one embodiment. In another embodiment, the control unit 12 changes the intensity I of the current, which flows through the heating element 11. These two embodiments may be combined with one another. Since the temperature of the semiconductor component 10 shall be above the ambient temperature, this one-sided temperature regulation with the heating element 11 as the final control element is sufficient. Even though possible, it is, however, usually unnecessary to cool the heating element 11 in a controlled manner.

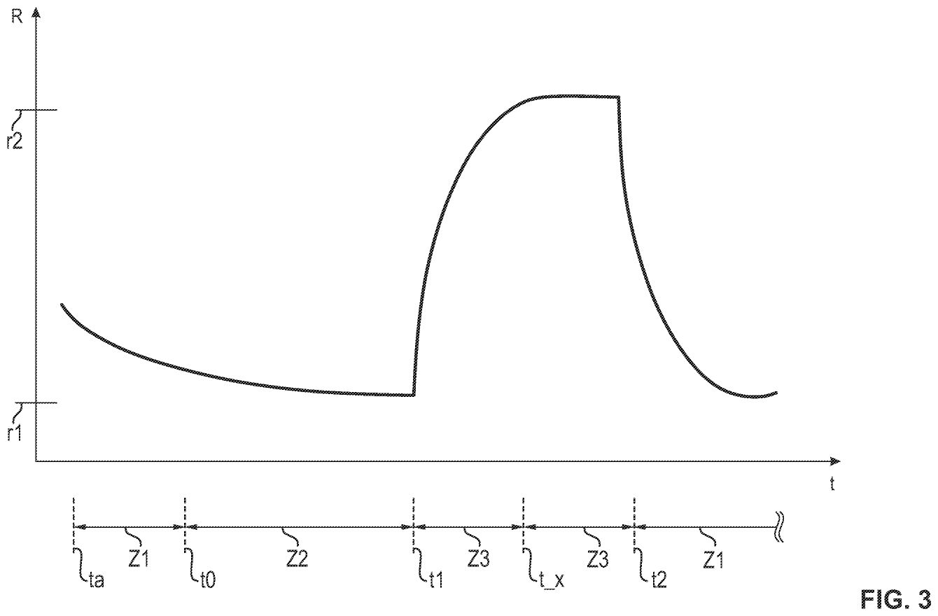

The electrical resistance of the semiconductor component 10 also depends on ambient conditions, especially on the oxygen content in the surrounding area, and at times also on the humidity, even in the case of approximately constant temperature. The electrical resistance R of the semiconductor component 10 is therefore measured at least at a first sampling time t1 and at a subsequent second sampling time t2. A measurement time period Z3 begins at the first sampling time t1, and this measurement time period Z3 ends at the second sampling time t2, cf. FIG. 3. It is possible to additionally measure the electrical resistance R at least once at a sampling time t_x between these two sampling times t1 and t2. This measurement time period Z3 is identical in the exemplary embodiment to an oxidation time period described below. Both the measurement time period and the oxidation time period begin at the first time t1 and end at the second time t2. The reference symbol Z3 is therefore also used for the oxidation time period. The first time t1 acts as the detection time and the second time t2 as the reference time in the exemplary embodiment.

In the view shown in FIG. 3, the time t is plotted on the x axis and the measured resistance R of the semiconductor component 10 on the y axis. The concentration of the combustible target gas in the gas sample G and hence in the measuring chamber 9 are identical with a sufficient accuracy to the target gas concentration in the area surrounding the gas detection device 100, i.e., in the area to be monitored. There is practically no combustible target gas in the measuring chamber 9 at the second sampling time (reference time) t2 because existing target gas was oxidized during the time period Z3. The measurement at the second sampling time t2 consequently acts as a reference measurement or as a zero-point measurement.

In order to make it possible to perform such a reference measurement at the second sampling time t2, the combustible target gas in the measuring chamber 9 is eliminated during the oxidation time period Z3, so that no combustible target gas is present in the measuring chamber 9 at the second time t2. This elimination is carried out by an oxidation component (oxidizer) 2 oxidizing the combustible target gas, which is present as a component of the gas sample G in the measuring chamber 9. It is, of course, possible that no combustible target gas is present in the area to be monitored and the measuring chamber 9 is therefore already free of combustible target gas at the first time t1.

A concentration threshold is preferably predefined as the upper threshold for the target gas concentration to be expected in the area to be monitored. This concentration threshold as well as the construction-related volume of the measuring chamber 9 determine the maximum possible quantity of combustible target gas in the measuring chamber 9. This maximum possible quantity of target gas is so small that sufficient oxygen is present in the measuring chamber 9 to oxidize the total quantity of target gas in the measuring chamber 9.

The oxidation time period Z3 is so long, on the one hand, that the total amount of combustible target gas in the measuring chamber 9 is oxidized in the course of the oxidation time period Z3, providing that the concentration of the target gas is below the concentration threshold. On the other hand, the oxidation time period Z3 is preferably as short as possible in order to make it possible to repeat the steps of oxidizing combustible target gas at the highest possible frequency and to measure the electrical resistance R of the semiconductor component 10 twice.

In one variant, a concentration threshold is not necessarily predefined. The measurement time period and oxidation time period Z3 is ended when measurements yield the result that the electrical resistance R remains constant; more precisely, when the slope of the time course of the electrical resistance R remains below a predefined threshold. This indicates that all the combustible target gas or at least a predefined percentage of the target gas in the measuring chamber 9 is oxidized.

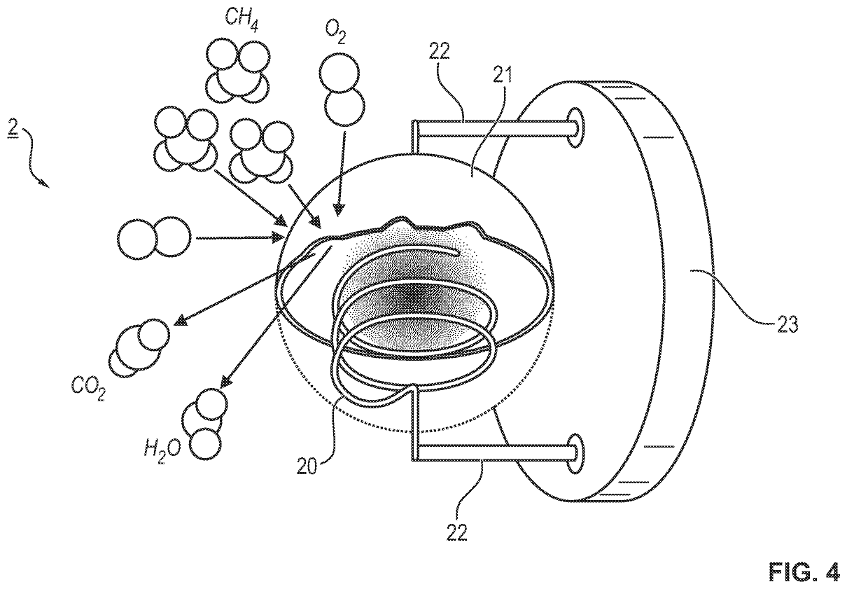

The combustible target gas in the measuring chamber 9 is oxidized by the oxidation component 2. FIG. 4 shows a preferred embodiment of the oxidation component 2. The combustible target gas is methane (CH₄) in this example. The oxidation component 2 brings about the chemical reaction

CH₄+2*O₂→2*H₂O+CO₂.

This chemical reaction is suggested in FIG. 4. Even though oxygen is bound during the oxidation of the target gas, the optional concentration threshold is predefined such that the reduction of the content of O₂ molecules in the measuring chamber 9 is so low based on the oxidation that the electrical resistance R of the semiconductor component 10 is changed thereby only negligibly.

In the preferred embodiment shown in FIG. 4, the oxidation component 2 is configured as a pellistor and comprises
a helical heating segment 20,
a preferably spherical jacketing 21 around the heating segment 20,
two electrical contacts 22 and
a plate 23.

An electrical voltage is applied to the heating segment 20. As a result, the heating segment 20 is heated to an operating temperature that is between 300° C. and 700° C. and preferably between 400° C. and 550° C. The heating segment 20 is in thermal contact with the jacketing 21, so that the jacketing 21 is heated as well.

However, this temperature alone would not yet be sufficient to oxidize combustible target gas to a sufficient extent. A higher temperature consumes more electrical energy and increases the risk that a target gas in the measuring chamber 9 will burn abruptly or even explode. In order to nevertheless oxidize all combustible target gas in the measuring chamber 9 at a temperature that is preferably below 550° C., a catalytic material, for example, platinum or a platinum oxide, is embedded in the jacketing 21. The jacketing 21 is preferably porous, so that the thermally acting surface of the jacketing 21 is larger than in the case of a smooth surface.

A thermal barrier 4, which is shown schematically in FIG. 1 and FIG. 2, is arranged between the semiconductor sensor 1 and the oxidation component 2. This thermal barrier 4 reduces the thermal effect of the oxidation component 2 on the semiconductor sensor 1 and therefore reduces the risk of the electrical resistance R of the semiconductor component 10 being changed appreciably by the heated oxidation component 2 and/or by the steps of switching on and by the switching off of the oxidation component 21, which could lead to an incorrect measurement. However, at least one opening, preferably a circular opening, is formed between the housing 5 and the thermal barrier 4, so that the gas sample G can flow through the entire measuring chamber 9. This is desirable for the oxidation component 2 to be able to oxidize the total amount of target gas in the measuring chamber 9, even the target gas behind the thermal barrier 4, and in order for the measured electrical resistance R of the semiconductor component 10 to be able to be used as an indicator for the target gas concentration being sought.

The control unit 12 is capable of switching on and switching off the oxidation component 2. In a first embodiment of the present invention the control unit 12 causes the oxidation component 2 to be in the switched-on state in each oxidation time period Z3 and in the switched-off state outside of an oxidation time period Z3. Since the oxidation component 2 has a relatively low thermal mass, it reaches the operating temperature between 300° C. and 700° C. rapidly after being switched on and cools rapidly to the temperature in the measuring chamber 9 after being switched off. This intentionally great and usually oscillating temperature change inherently leads, as a rule, to an oscillating input of thermal energy per unit of time by the oxidation component 2 to the outer surface of the semiconductor sensor 1. The oscillating temperature cause, as a rule, a change in the electrical resistance R of the semiconductor component 10 and could therefore lead to incorrect measurements.

The oxidation component 2, that has a temperature that oscillates in the first embodiment, may have an undesired thermal effect on the semiconductor component 10. In order to reduce this thermal effect in the measuring chamber 9, an actuatable heating element 3 is additionally arranged in the measuring chamber 9, specifically on the same side of the thermal barrier 4 as the oxidation component 2.

According to the first embodiment, the control unit 12 is capable of switching on and switching off not only the oxidation component 2 but also the heating element 3. The heating element 3 in the switched-on state ideally brings about the same input of thermal energy per unit of time into the semiconductor component 10 as the oxidation component 2 in the switched-on state. The effect is that the oxidation component 2 has a similar thermal effect on the semiconductor component 10 as the heating element 3. The risk that the current temperature of the oxidation component 2 will distort the measurement results of the semiconductor sensor 1 is reduced.

In one embodiment, the heating element 3 comprises, just as the oxidation component 2, a helical heating segment 20, a jacketing 21 and electrical contacts 22, but no catalytic material in the jacketing 21. Therefore the heating element 3 in the switched-on state is incapable of oxidizing combustible target gas in the measuring chamber 9 even with the heating segment 20 heating.

There is an inlet time period Z1 chronologically before or even after the oxidation time period (=measurement time period) Z3. A gas sample G can flow at least during this inlet time period Z1 from the area into the measuring chamber 9, especially by the gas sample G being suctioned by the pump 13 and/or by diffusing into the measuring chamber 9. There is a heating time period Z2 between the inlet time period Z1 and the oxidation time period Z3, cf. FIG. 3.

During each oxidation time period Z3 the oxidation component 2 is in the switched-on state and the heating element 3 is in the switched-off state. During the heating time period Z2, the heating element 3 is in the switched-on state and the oxidation component 2 is in the switched-off state. The heating element 3 heats the gas sample G in the measuring chamber 9, so that no abrupt temperature change occurs in the measuring chamber 9 during the transition from a heating time period Z2 to an oxidation time period (=measurement time period Z3). Thanks to the heating element 3, the input of thermal energy per unit of time into the semiconductor component 10 during the heating time period Z2 is approximately equal to the input occurring during the measurement time period Z3. In particular, the input of heating energy into the semiconductor sensor 1 varies less over time compared to a state without a heating element 3. Preferably both the oxidation component 2 and the heating element 3 are in the switched-off state during the inlet time period Z1.

An adjustment is carried out in advance in one embodiment in order to set a desired operating temperature Temp_Soll(3) of the heating element 3. The heating element 3 reaches this desired operating temperature Temp_Soll(3) after being switched on. The goal of the adjustment is that the input of thermal energy per unit of time on to the semiconductor sensor 1, which input is brought about by the heating element 3 in the switched-on state, is equal to the input of thermal energy per unit of time that is brought about by the oxidation component 2 in the switched-on state. Aside from the desired operating temperature Temp_Soll(3), the distance dist(3) between the heating element 3 and the semiconductor sensor 1 as well as the distance dist(2) between the oxidation component 2 and the semiconductor sensor 1 can be changed as well. A state in which the measuring chamber 9 is free from combustible target gas is established for the adjustment. The desired operating temperature Temp_Soll(3) and the distance are set such that the detection variable, i.e., here the electrical resistance R of the semiconductor component 10 with the oxidation component 2 in the switched-on state and with the heating element 3 in the switched-off state, is exactly the same as when the oxidation component 2 is in the switched-off state and the heating element 3 is in the switched-on state.

FIG. 3 illustrates an exemplary time course during the operation of the gas detection device 100. The time t is plotted on the x axis and the electrical resistance R of the semiconductor component 10 on the y axis. The following description pertains to the first embodiment according to FIG. 1.

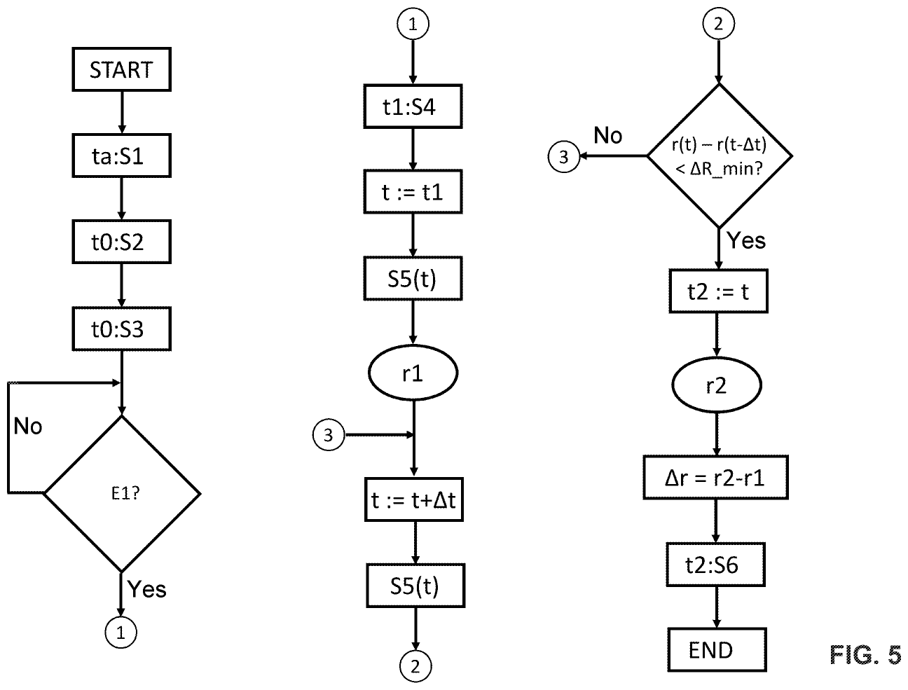

A sequence which comprises the inlet time period Z1, the subsequent heating time period Z2 and the subsequent measurement time period (=oxidation time period Z3) is carried out at least once. This sequence with the three time periods Z1, Z2, Z3 is preferably carried out repeatedly, while the gas detection device 100 is used. FIG. 5 shows an exemplary flow chart for such a sequence during the operation of the gas detection device 100.

The inlet time period Z1 begins at the time ta. During the inlet time period Z1 the valves 6 and 7 are opened and the optional pump 13 is switched on. The measuring chamber 9 is flushed and filled with a new gas sample G. This means that the gas sample G that was present in the measuring chamber 9 before flows out of the measuring chamber 9 through the outlet A and the gas sample G now being tested flows out of the area to be monitored through the inlet E into the measuring chamber 9. In one embodiment, the optional pump 13 is in a switched-on state and delivers the gas sample G from the surrounding area into the measuring chamber 9. It is also possible that the gas sample G to be analyzed diffuses from the area into the measuring chamber 9.

In FIG. 5, ta:S1 denotes the step in which the valves 6 and 7 are opened at the time ta, the pump 13 is switched on and the measuring chamber 9 is flushed. The heating element 3 and the oxidation component 2 are in the switched-off state.

The inlet time period Z1 is so long that the concentration of combustible target gas in the measuring chamber 9 is approximately equal to the target gas concentration in the surrounding area and therefore in the area to be monitored after the end of the inlet time period Z1. In particular, the inlet time period Z1 is so long that when no combustible target gas is detected in the measuring chamber 9, it is certain that no combustible target gas above a detection limit is present in the surrounding area, either. In one embodiment, the duration of the inlet time period Z1 is set at a fixed value.

During the inlet time period Z1 the oxidation component 2 is in the switched-off state. In one embodiment, during the inlet time period Z1 the heating element 3 is also in the switched-off state, as a result of which electrical energy is saved. In another embodiment, the heating element is switched on or is in the switched-on state during the inlet time period Z1, which often makes a shorter heating time period Z2 possible and thereby saves time.

The inlet time period Z1 terminates at the time t0, and the subsequent heating time period Z2 begins. During the heating time period Z2 the oxidation component 2 remains in the switched-off state. At the time t0 the control unit 12 triggers the following events:
The valves 6 and 7 are closed, so that the measuring chamber 9 is separated from the surrounding area.
The optional pump 13 is switched off.
The heating element 3 is switched on.

In FIG. 5, t0:S2 denotes the step in which the valves 6 and 7 are closed at the time t0 and the pump 13 is switched off. t0:S3 denotes the step in which the heating element 3 is switched on at the time t0. The oxidation component 2 remains in the switched-off state.

The heating time period Z2, during which the heating element 3 is in the switched-on state, is of a length that during the heating time period Z2 the heating element 3 is heated to the desired operating temperature Temp_Soll(3). The operating temperature Temp_Soll(3) was determined, as was described above, during a previous adjustment and brings about the same input of thermal energy per unit of time as the oxidation component 2 in the switched-on state will do later.

In one embodiment, the desired operating temperature Temp_Soll(3) of the heating element 3 is predefined. The current temperature Temp(3) of the heating element 3 is measured. For example, the current electrical resistance of the heating element 3 is measured. The electrical resistance of a metal is known to be correlated with the temperature of that metal, so that the resistance is an indicator for the temperature.

In FIG. 5, E1? denotes the decision on whether the heating time period Z2 has already come to an end or not, i.e., whether the heating element has reached the desired operating temperature Temp_Soll(3).

The heating time period Z2 is ended at the time t1 (detection time), and the measurement time period Z3 begins. The control unit 12 triggers the following events at the time t1:
The electrical resistance R of the semiconductor component 10 is measured. The value of the electrical resistance measured at t1 is designated by r1.
The heating element 3 is switched off.
The oxidation component 2 is switched on.

During the measurement time period Z3 the heating element 3 remains in the switched-off state. The valves 6 and 7 remain closed, and the pump 13 remains in the switched-off state. The oxidation component 2, which is switched on during the oxidation time period (=measurement time period) Z3, oxidizes the combustible target gas or each combustible target gas in the measuring chamber 9. It is, of course, possible that no combustible target gas is present in the area to be monitored and hence no oxidation is carried out in the measuring chamber 9 and the heated oxidation component 2 does not carry out any oxidation.

In the exemplary embodiment the first time t1 is both the end of the heating time period Z2 and the beginning of the measurement time period (=oxidation time period) Z3. In FIG. 5, t1:S4 denotes the step that the heating element 3 is switched off and the oxidation component 2 is switched on at the time t1. S5(t) denotes that the electrical resistance R of the semiconductor component 10 is measured at the time t. The time t is set initially at t1. The measurement S5(t1) performed at the first time t1 yields the resistance value r1.

The measurement time period Z3 and hence the sequence comprising the time periods Z1, Z2, Z3 terminate at the time t2 (reference time).

The control unit 12 triggers the following events at the time t2:
The electrical resistance R of the semiconductor component 10 is measured again. The measurement S5(t2) at the second time t2 yields a value of the electrical resistance R, which is designated by r2.
The oxidation component 2 is switched off.

The oxidation time period Z3 between t1 and t2 is not predefined as a fixed value in the exemplary flow chart. The electrical resistance R is rather measured at the times t1, t1+Δt, t1+2*Δt, . . . , wherein Δt is a distance predefined at a fixed value. Since the oxidation component 2 burns combustible target gas in the measuring chamber 9, the electrical resistance R increases steadily. The resistance value at the time t is designated by r(t). The difference between the resistance values at two times t−Δt and t directly following one another, i.e., the difference r(t)−r(t−Δt), is calculated.

No combustible target gas can enter into the measuring chamber 9 from the outside during the measurement time period Z3 in the example shown. If the difference r(t)−r(t−Δt) is smaller than a predefined threshold ΔR_min, practically all combustible target gas present in the measuring chamber 9 has been oxidized. The time t at which this is determined is used as the time t2, at which the measurement time period Z3 is ended. The resistance value r(t) measured most recently is used as the value r2=r(t2).

The difference r(t)−r(t−Δt) between the two most recent resistance values is used in the example shown. In general, the slope of the electrical resistance R is calculated as a function of time, for which the time series r(t1), r(t1+Δt), r(t1+2*Δt), . . . is used. When this slope is below a predefined threshold, the time of the most recent measurement is used as the second time t2.

In FIG. 5, t2:S6 denotes that the oxidation component 2 is switched off at the time t2.

A next sequence begins. The inlet time period Z1 of the next sequence is suggested in FIG. 3. The valves 6 and 7 are optionally opened again and the pump 13 is switched on again at the second time t2.

In the first embodiment just described, the oxidation component 2 is only during the oxidation time period Z3 in the switched-on state and is otherwise in the switched-off state. An alternative second embodiment will be described below. In this second embodiment the oxidation component 2 is not only during the oxidation time period Z3 in the switched-on state, but at least also during the inlet time period Z1. The oxidation component 2 optionally remains in the switched-on state during the entire operation of the gas detection device 100 and is only in a resting state of the gas detection device 100 in the switched-off state. The embodiment in which the oxidation component 2 is left in the switched-on state eliminates in many cases the need for a heating element 3. The inlet time period Z1 may be followed directly by the oxidation time period Z3, i.e., a heating time period Z2 is not needed. However, the second embodiment may also be used combined with a heating element 3, which compensates to a certain degree possible fluctuations in the input of thermal energy by the oxidation component 2 onto the semiconductor component 10.

The measuring chamber 9 is opened during the inlet time period Z1 in the second embodiment, so that a gas sample G can flow from the area to be monitored into the measuring chamber 9. The measuring chamber 9 is closed and is thus sealed in a fluid-tight manner against the area to be monitored during the oxidation time period Z3, so that no target gas can flow into the measuring chamber 9 during the oxidation time period Z3, even if combustible target gas is present in the area.

The process of opening and closing the measuring chamber 9 and thereby operating it optionally in an open state or in a closed state can be embodied, for example, in the following manners:
The pump 13 is switched on and switched off. In particular, the pump 13 is switched on during the inlet time period Z1 and is switched off during the oxidation time period.
The valve 6 at the inlet E is opened and closed. The valve 6 is open during the inlet time period Z1 and is closed during the oxidation time period Z3.
The above-described perforated orifice or even a flap is moved relative to the housing 5, so that the perforated orifice or the flap releases the inlet E during the inlet time period Z1 and blocks the inlet E during the oxidation time period Z3.

These two embodiments may be combined with one another, which increases in many cases the reliability of the gas detection device 100. According to this combination, The inlet E is in the opened state and the oxidation component 2 and the optional heating element 3 are in the switched-off state during the inlet time period Z1. During the oxidation time period Z3 the inlet E is in the closed state, the oxidation component 2 is in the switched-on state and the optional heating element 3 is in the switched-off state. During the optional heating time period Z2 the inlet E is preferably in the closed state.

The following description pertains to the embodiment according to FIG. 1 and to both embodiments, i.e., both to the embodiment in which the oxidation component 2 is switched on and is switched off and to the embodiment in which the measuring chamber 9 is opened and closed.

The two measured values r1 and r2 and optionally additional measured values for the electrical resistance R are transmitted to a signal-processing analysis unit 15, which is a part of the control unit 12 in the embodiment shown. As was already described, the electrical resistance R of the semiconductor component 10 decreases in the exemplary embodiment with increasing percentage (concentration) of combustible target gas in the measuring chamber 9. The electrical resistance R depends, in addition, on the ambient conditions. The difference Δr=r2−r1 is calculated and analyzed according to the present invention, specifically by the analysis unit 15. This difference At depends essentially only on the sought concentration of combustible target gas in the measuring chamber 9, while ambient conditions have approximately the same effect on the electrical resistance R at both measurement times t1 and t2. “Essentially” means that the effect of ambient conditions on the difference Δr is negligibly minor. The effect of ambient conditions is compensated by calculation in this manner.

In one embodiment, the gas detection device 100 is used for the decision on whether at least one combustible target gas is present in the area to be monitored or not. A sequence Z1, Z2, Z3 is carried out during a use. If the measured difference Δr after a sequence is above a difference threshold, a combustible target gas is detected. It is otherwise certain that no combustible target gas is currently present, assuming, of course, that the gas detection device 100 is intact. The inventors determined in internal experiments with a defined combustible target gas that a gas detection device 100 according to the present invention is capable of detecting this target gas with a concentration of less than 10 ppm in a reliable manner, often even at a concentration of less then 2 ppm.

The gas detection device 100 is preferably calibrated in advance. Different concentrations con(1), con(2), . . . of a target gas to be detected are established in the area surrounding the gas detection device 100 during this calibration. A difference Δr(i) is measured at least once for each concentration con(i). A plurality of sequences Z1, Z2, Z3 are preferably carried out, and averaging is carried out over the measured differences. The calibration yields an empirically determined functional relationship Con=f(AR). This empirically determined functional relationship is stored in a computer-evaluable form in a memory of the analysis unit 15. Note: Con designates the variable and con designates a defined measured value for this variable.

The heating element 3 is preferably also set, as described above, during this adjustment, so that the input of thermal energy per unit of time by the heating element 3 is equal to the input per unit of time by the oxidation component 2.

The sequence Z1, Z2, Z3 is carried out repeatedly during a use of the gas detection device 100. The stored functional relationship f is applied to the measured difference Δr and it yields the current target gas concentration con=f(Δr) sought.

The electrical resistance R of the semiconductor component 10 is measured according to the present invention at least at the two times t1 and t2. In one embodiment, the electrical resistance R is additionally measured at at least one intermediate time t_x, which is between the times t1 and t2. In a preferred variant, the time course of the electrical resistance R is measured during the measurement time period Z3. Freak values and other measurement errors are compensated by calculation up to a certain degree by suitable numerical methods, for example, by means of a smoothing.

In the second embodiment according to FIG. 2, the gas detection device 100 does not comprise any inlet E and any outlet A. In one embodiment, a displaceable diaphragm or another suitable closure is capable of optionally releasing or closing the fluid communication between the measuring chamber 9 and the surrounding area. At least one sequence with an inlet time period Z1, with a heating time period Z2 and with a measurement time period Z3 is likewise carried out in this embodiment, and the fluid communication between the measuring chamber 9 and the surrounding area is opened during the inlet time period Z1 and interrupted during the heating time period Z2 and the measurement time period Z3. The embodiment according to FIG. 2 and with the movable closure may be combined with an embodiment in which the oxidation component 2 is continuously in the switched-on state and even though a heating element 3 may be present, but it is not needed.

If, by contrast, a fluid communication is established continuously between the measuring chamber 9 and the surrounding area by means of the diaphragm 8, a sequence, which comprises only a heating tine period Z2 and a subsequent measurement time period Z3, is carried out preferably at least once, and the control unit 12 triggers the processes described above for these two time periods Z2 and Z3. The heating time period Z2 acts at the same time as the inlet time period Z1 in this embodiment.

While specific embodiments of the invention have been shown and described in detail to illustrate the application of the principles of the invention, it will be understood that the invention may be embodied otherwise without departing from such principles.

List of Reference Numbers

| | |
|---|---|
| 1 | Semiconductor sensor; it comprises the semiconductor component 10 |
| | and the heating element 11 |
| 2 | Actuatable oxidation component (oxidizer) 2; it comprises the hot |
| | wire 20, the ceramic and catalytic jacketing 21, the electrical |
| | contacts 22 and the plate 23; it is configured as a catalytic pellistor |
| 3 | Actuatable heating element in the measuring chamber 9 |
| 4 | Thermal barrier between the oxidation component 2 and the heating element |
| | 3, on the one hand, and the semiconductor sensor 1, on the other hand |
| 5 | Housing of the gas detection device 100; it encloses the measuring |
| | chamber 9 |
| 6 | Valve at the inlet E |
| 7 | Valve at the outlet A |
| 8 | Gas-permeable diaphragm; it connects the measuring chamber 9 to the |
| | surrounding area |
| 9 | Measuring chamber of the gas detection device 100; it enclosed by the |
| | housing 5; it accommodates the semiconductor sensor 1, the oxidation |
| | component 2, the heating element 3 and the thermal barrier 4 |
| 10 | Semiconductor component of the semiconductor sensor 1, whose electrical |
| | resistance R is measured; it acts as the sensor component |
| 11 | Heating element of the semiconductor sensor 1; it heats the semiconductor |
| | component 10 |
| 12 | Signal-processing control unit; it controls the heating element 11 of the |
| | semiconductor sensor 1, the oxidation component 2, the heating element 3, |
| | the optional valves 6 and 7 and the optional pump 13; it comprises the |
| | analysis unit 15 |
| 13 | Actuatable pump, which suctions a gas sample G from the surrounding |
| | area and feeds it through the inlet E into the measuring chamber 9 |
| 14 | System clock of the control unit 12 |
| 15 | Signal-processing analysis unit; it determines the concentration of the |
| | combustible target gas; it is a component of the control unit 12 |
| 20 | Hot wire of the oxidation component 2 |
| 21 | Ceramic and catalytic jacketing of the oxidation component 2 |
| 22 | Electrical contacts of the oxidation component 2 |
| 23 | Plate of the oxidation component 2 |
| 24 | Current intensity sensor; it measures the intensity I of the current, which |
| | flows through the semiconductor component 10 |
| 25 | Voltage sensor; it measures the electrical voltage U, which is present at the |
| | semiconductor component 10 |
| 100 | Gas detection device; it comprises the measuring chamber 9 in the |
| | housing 5, the semiconductor sensor 1, the oxidation component 2, the |
| | heating element 3, the thermal barrier 4, optionally the inlet E with the |
| | valve 6 and the outlet A with the valve 7, optionally the diaphragm 8 and |
| | optionally the pump 13 |
| A | Outlet from the measuring chamber 9; it comprises the valve 7 |
| dist(2) | Distance between the oxidation component 2 and the semiconductor |
| | sensor 1 |
| dist(3) | Distance between the heating element 3 and the semiconductor sensor 1 |
| E | Inlet to the measuring chamber 9; it comprises the valve 6 |
| E1? | Decision: Heating time period Z2 elapsed? |
| G | Gas sample from the area to be monitored; it flows through the inlet E or |
| | the diaphragm 8 into the measuring chamber 9 and is tested there |
| R | Electrical resistance of the semiconductor component 10; it is determined |
| | on the basis of measured values of the sensors 24 and 25 |
| r1 | Measured value at the time t1 for the electrical resistance R |
| r2 | Measured value at the time t2 for the electrical resistance R |
| r(t) | Measured value at the time t for the electrical resistance R |
| ΔR_min | Predefined threshold for the change in the electrical resistance R |
| S1 | Step: The valves 6 and 7 are opened, the pump 13 is switched on, and the |
| | measuring chamber 9 is flushed at the time ta |
| S2 | Step: The valves 6 and 7 are closed and the pump 13 is switched on at the |
| | time t0 |
| S3 | Step: The heating element 3 is switched on at the time t0 |
| S4 | Step: The heating element 3 is switched off and the oxidation component |
| | 2 is switched on at the time t1 |
| t0 | Beginning of the heating time period Z2 |
| t1 | First time at which the detection variable is measured and which acts as the |
| | detection time - combustible target gas may be present in the measuring |
| | chamber 9 at the first time |
| t2 | Second time, at which the detection variable is measured and which acts as |
| | the reference time - no combustible target gas is present in the measuring |
| | chamber 9 due to the oxidation at the second time |
| ta | Beginning of the inlet time period Z1 |
| Z1 | Inlet time period, during which the gas sample G flows into the measuring |
| | chamber 9 and the heating element 3 is in the switched-off state |
| Z2 | Heating time period, during which the heating element 3 is in the switched- |
| | on state and the oxidation component 2 is in the switched-off state; it begins |
| | at the time t0 |
| Z3 | Oxidation time period, during which the oxidation component 2 is switched |
| | on and the heating element 3 is in the switched-off state; it extends from |
| | the first time t1 to the second time t2 |

What is claimed is:

1. A gas detection device for monitoring a spatial area for a combustible target gas, the gas detection device comprising:
a measuring chamber, wherein the gas detection device is configured such that a gas sample flows at least from time to time from the area into the measuring chamber;
an electrically conductive sensor, wherein the gas detection device is configured such that the electrically conductive sensor comes into contact with the gas sample in the measuring chamber, the electrically conductive sensor having a detection variable, which detection variable changes with decreasing concentration of the combustible target gas in the gas sample present in the measuring chamber;
an oxidizer configured to oxidize combustible target gas that is contained in a gas sample in the measuring chamber, wherein the gas detection device is configured such that the oxidizer oxidizes combustible target gas in the gas sample in the measuring chamber completely or at least partially during an oxidation time period;
a detection sensor configured to measure an indicator for the detection variable of the electrically conductive sensor both at a detection time and at a reference time, wherein the oxidation time period begins at or after the earlier time of the detection time and the reference time and ends before or at the later time of the detection time and the reference time; and
a signal-processing analysis unit configured to calculate a difference between the indicator value measured at the reference time and the indicator value measured at the detection time for the detection variable and, depending on the difference, to decide whether a combustible target gas is present in the gas sample or not, and/or to determine a concentration of the combustible target gas in the gas sample,
wherein, if combustible target gas is present in the area to be monitored, combustible target gas is also present at the detection time in the measuring chamber and based on the oxidation taking place during the oxidation time period, less or even no combustible target gas is present in the measuring chamber at the reference time than at the detection time.

2. A gas detection device in accordance with claim 1, further comprising a heating element configured to be switched on and to be switched off and configured to heat the gas sample in the measuring chamber in a switched-on state, wherein:
the oxidizer is configured to be switched on and to be switched off;
the gas detection device is configured such that the heating element is in the switched-on state and the oxidizer is in a switched-off state during a heating time period; and
the gas detection device is configured such that the oxidizer is in a switched-on state and the heating element is in a switched-off state during the oxidation time period.

3. A gas detection device in accordance with claim 2, wherein:
the oxidizer in the switched-on state brings about an input of thermal energy into the electrically conductive sensor and the heating element in the switched-on state brings about an input of thermal energy into the electrically conductive sensor; and
the input of thermal energy per unit of time by the heating element in the switched-on state is equal to the input of thermal energy per unit of time by the oxidizer in the switched-on state.

4. A gas detection device in accordance with claim 2, wherein the gas detection device is configured such that under identical ambient conditions and under the absence of target gas the detection variable of the electrically conductive sensor assumes the same value when the oxidizer is in the switched-on state and the heating element is switched-off state as when the oxidizer is in the switched-off state and the heating element is in the switched-on state.

5. A gas detection device in accordance with claim 2, wherein:
the gas detection device is configured such that the heating time period chronologically precedes the oxidation time period; and
an end of the heating time period is coordinated with a beginning of the oxidation time period.

6. A gas detection device in accordance with claim 1, wherein the oxidizer is configured to be switched on and to be switched off and is in a switched-on state during the oxidation time period and is in a switched-off state outside of the oxidation time period.

7. A gas detection device in accordance with claim 1, wherein:
the gas detection device is configured to carry out at least once a slope calculation sequence;
the slope calculation sequence comprises the steps of:
the detection sensor measuring the indicator for the detection variable of the electrically conductive sensor at at least two times, providing at least two measured values, wherein the two times are within the oxidation time period and are chronologically at different times; and
the analysis unit calculating an indicator for the slope of the detection variable over time depending at least on the two measured values of the detection variable; and
the analysis unit is configured to use the chronologically most recent time at which the indicator for the detection variable was measured as the reference time and the measured value at the chronologically most recent time as the measured value at the reference time when the slope calculated in a slope calculation sequence is below a predefined threshold.

8. A gas detection device in accordance with claim 1, wherein
the oxidizer is configured to be switched on and to be switched off;
the oxidizer is in a switched-on state during the oxidation time period and is in a switched-off state at least during an inlet time period; and
the gas detection device is configured such that the gas sample flows from the spatial area into the measuring chamber at least during the inlet time period.

9. A gas detection device in accordance with claim 8, wherein the gas detection device is configured such that the gas sample also flows from the spatial area into the measuring chamber during the oxidation time period.

10. A gas detection device in accordance with claim 1, wherein:
the measuring chamber is configured to be selectively operated in an open state and in a closed state;
the gas detection device is configured such that the gas sample flows into the measuring chamber in the open state and the measuring chamber is fluid-tightly sealed against the spatial area in the closed state;
the gas detection device is configured such that the measuring chamber is in the open state during an inlet time period, which inlet time period comprises the detection time or is chronological before the detection time; and
the gas detection device is configured such that the measuring chamber is in the closed state at the reference time.

11. A gas detection device in accordance with claim 10, wherein the gas detection device is configured such that the oxidizer is in a switched-on state during the inlet time period.

12. A gas detection device in accordance with claim 1, wherein electrical current flows through the electrically conductive sensor at least when the oxidizer is in a switched-on state.

13. A gas detection device in accordance with claim 1, wherein the oxidizer comprises the electrically conductive sensor.

14. A gas detection device in accordance with claim 1, wherein the oxidizer is configured as a pellistor and comprises a heating element, the heating element operating as the electrically conductive sensor, and the oxidizer comprises a ceramic jacketing around the heating element and a catalytic coating on the ceramic jacketing or a catalytic admixture in the ceramic jacketing for oxidizing to oxidize the combustible target gas.

15. A process for monitoring a spatial area for a combustible target gas, the process comprising the steps of:
providing a gas detection device, which comprises: a measuring chamber; an electrically conductive sensor; a detection sensor and an oxidizer;
causing or allowing a gas sample to flow at least temporarily from the spatial area into the measuring chamber;
causing or allowing the gas sample in the measuring chamber to come into contact with the electrically conductive sensor, wherein the contact has an effect on the electrically conductive sensor such that a measurable detection variable of the electrically conductive sensor changes with decreasing concentration of the combustible target gas in the gas sample present in the measuring chamber;
with the oxidizer, oxidizing combustible target gas, which is contained in the gas sample in the measuring chamber, at least during an oxidation time period;
with the detection sensor, measuring a respective value of an indicator for the detection variable both at a detection time and at a reference time, wherein the oxidation time period begins at or after the earlier time of the detection time and the reference time and ends before or at the later time of the detection time and the reference time, wherein, when combustible target gas is present in the area, combustible target gas is also present in the measuring chamber at the detection time and less or even no combustible target gas is present in the measuring chamber at the reference time than at the detection time, the effect is achieved thanks to oxidation taking place during the oxidation time period;
calculating a difference between the measured indicator value at the reference time and the measured indicator value at the detection time; and
depending on the difference, deciding whether or not a combustible target gas is present in the gas sample, and/or determining a concentration of the combustible target gas in the gas sample.

16. A process in accordance with claim 15,
wherein the gas detection device further comprises a heating element configured to be switched on and to be switched off,
wherein the oxidizer is configured to be switched on and to be switched off,
wherein the process further comprising the steps of:
switching on the oxidizer at the beginning of the oxidation time period and switching off the oxidizer at the end of the oxidation time period;
switching on the heating element at the beginning of a heating time period, which is outside of the oxidation time period, and switching off the heating element at the end of the heating time period,
wherein with the heating element in a switched-on state, heating the gas sample in the measuring chamber.

17. A process in accordance with claim 16, wherein:
wherein the heat input per unit of time into the electrically conductive sensor effected by the heating element in the switched-on state is equal to the heat input per unit of time effected by the oxidizer in a switched-on state.

18. A process in accordance with claim 15, wherein:
a slope calculation sequence is carried out at least once,
the slope calculation sequence comprises the following steps:
the detection sensor measures the indicator for the detection variable of the electrically conductive sensor at at least two times, wherein the two times are within the oxidation time period and are chronologically spaced apart from one another; and
an indicator for the slope of the detection variable over time is calculated depending at least on the measured values of the detection variable; and
if the calculated slope is below a predefined threshold, the chronologically most recent time, at which the indicator for the detection variable was measured, is used as the reference time and the measured value at the chronologically most recent time is used as the measured value at the reference time.

19. A process in accordance with claims 15,
wherein the oxidizer is configured to be switched on and to be switched off,
wherein a first sequence and/or a second sequence is carried out,
wherein the first sequence comprises the steps of:
operating the oxidizer in a switched-off state during an inlet time period, which comprises the detection time or is before the detection time; and
causing or allowing the gas sample to flow into the measuring chamber at least during the inlet time period; and
wherein the second sequence comprises the steps of:
operating the measuring chamber during the inlet time period in an open state, in which the gas sample flows from the spatial area to be monitored into the measuring chamber;
operating the measuring chamber in a closed state, in which the measuring chamber is fluid tightly sealed against the area, wherein the measuring chamber is operated in the open state or is changed over from the closed state into the open state at the detection time; and
operating the oxidizer in the switched-on state at least when the measuring chamber is operated in the closed state.

20. A process in accordance with claim 15, wherein the oxidizer comprises the electrically conductive sensor.

21. A process in accordance with claim 20, wherein the oxidizer is configured as a pellistor and comprises a heating element, the heating element operating as the electrically conductive sensor, and the oxidizer comprises a ceramic jacketing around the heating element and a catalytic coating on the ceramic jacketing or a catalytic admixture in the ceramic jacketing for oxidizing to oxidize the combustible target gas.
